(19) United States
(12) Patent Application Publication
(10) Pub. No.: US 2011/0264030 A1
(43) Pub. Date: Oct. 27, 2011

(54) DELIVERY APPARATUS AND ASSOCIATED METHOD

(75) Inventors: Joseph M. Desimone, Chapel Hill, NC (US); Lukas Miller Roush, Chapel Hill, NC (US); Elizabeth Marti Enlow, Chapel Hill, NC (US); Mary Elizabeth Napier, Carrboro, NC (US)

(73) Assignee: The University of North Carolina At Chapel Hill, Chapel Hill, NC (US)

(21) Appl. No.: 12/918,916

(22) PCT Filed: Feb. 25, 2009
(86) PCT No.: PCT/US09/35070

§ 371 Date: Nov. 23, 2010

Related U.S. Application Data
(60) Provisional application No. 61/031,083, filed on Feb. 25, 2008.

(51) Int. Cl. A61M 37/00; A61N 1/30

(52) U.S. Cl. 604/21; 604/503

(57) ABSTRACT

A delivery apparatus for delivering a cargo to a target site of internal body tissue is provided. The delivery apparatus comprises a flexible tubular member having a distal end adapted for insertion proximate to the target site. A first electrode is configured for insertion within the flexible tubular member such that the first electrode is capable of being disposed proximate to the target site. A second electrode is configured to cooperate with the first electrode to form an electric field. A delivery component has a cargo carried thereby and is coupled with the first electrode. The delivery component is configured to degrade when exposed to the electric field such that the cargo is released to the target site upon degradation thereof. An associated method is also provided.

### FEDERALLY SPONSORED RESEARCH OR DEVELOPMENT

This disclosure was partially made with U.S. Government support under contract number CHE-9876674 awarded by the United States National Science Foundation (NSF). The U.S. Government may have certain rights in the disclosure.

### BACKGROUND

1. Field of the Invention

Embodiments of the present invention relate to a delivery apparatus, and more particularly, to an apparatus for facilitating delivery of various cargos to target sites and an apparatus associated therewith, wherein the apparatus provides an electric field to drive cargo through tissue as in iontophoretic approaches or where the apparatus induces the electrochemical degradation of a delivery component to release the various cargos, and combinations thereof.

2. Description of Related Art

Many techniques exist for the delivery of drugs and therapeutic agents to the body. Traditional delivery methods include, for example, oral administration, topical administration, intravenous administration, and intramuscular, intradermal, and subcutaneous injections. With the exception of topical administration which permits more localized delivery of therapeutic agents to particular area of the body, the aforementioned drug delivery methods generally result in systemic delivery of the therapeutic agent throughout the body. Accordingly, these delivery methods are not appropriate for localized targeting of drugs and therapeutic agents to specific internal body tissues.

As a result, other methods, such as endovascular medical devices, Natural Orifice Translumenal Endoscopic Surgery (NOTES)-based devices, and iontophoresis, have been developed to provide localized targeting of therapeutic agents to a particular internal body tissue. Iontophoresis is a form of drug delivery that uses electrical current to enhance the movement of charged molecules across or through tissue. Iontophoresis is usually defined as a non-invasive method of propelling high concentrations of a charged substance, normally therapeutic or bioactive-agents, transdermally by repulsive electromotive force using a small electrical charge applied to an iontophoretic chamber containing a similarly charged active agent and its vehicle. In some instances, one or two chambers are filled with a solution containing an active ingredient and its solvent, termed the vehicle. The positively charged chamber (anode) repels a positively charged chemical, while the negatively charged chamber (cathode) repels a negatively charged chemical into the skin or other tissue. Unlike traditional transdermal administration methods that involve passive absorption of a therapeutic agent, iontophoresis relies on active transportation within an electric field. In the presence of an electric field, electromigration and electroosmosis are the dominant forces in mass transport. As an example, iontophoresis has been used to treat the dilated vessel in percutaneous transluminal coronary angioplasty (PTCA), and thus limit or prevent restenosis. In PTCA, catheters are inserted into the cardiovascular system under local anesthesia and an expandable balloon portion is then inflated to compress the atherosclerosis and dilate the lumen of the artery.

The delivery of drugs or therapeutic agents by iontophoresis avoids first-pass drug metabolism, a significant disadvantage associated with oral administration of therapeutic agents. When a drug is taken orally and absorbed from the digestive tract into the blood stream, the blood containing the drug first passes through the liver before entering the vasculature where it will be delivered to the tissue to be treated. A large portion of an orally ingested drug, however, may be metabolically inactivated before it has a chance to exert its pharmacological effect on the body. Furthermore it may be desirable to avoid systematic delivery all together in order to allow high doses locally while avoiding potential side effects elsewhere, wherein local delivery is desirable for localized conditions. Existing medical device technologies that enable localized placement of therapeutics fail to provide the opportunity to embed/secure therapeutics in the tissue(s) of interest.

Accordingly, it would be desirable to provide an improved apparatus and method for selectively and locally targeting delivery of various drugs and therapeutic agents to an internal body tissue, and fixing such cargos in the tissue(s) of interest. Further, it would be desirable to provide an apparatus and method for delivering various drugs and therapeutic agents to a bodily segment removed from a patient for external treatment thereof.

### SUMMARY

The present invention relates to a delivery apparatus and method, and in particular, a delivery apparatus adapted for delivering a cargo to a target site of body tissue. The delivery apparatus comprises a flexible tubular member having a distal end adapted for insertion proximate to a target site of internal body tissue. A first electrode is configured to extend within the flexible tubular member so as to be disposed proximate to the target site. A second electrode is in electrical communication with the first electrode and is opposably positionable with respect thereto. The second electrode is configured to cooperate with the first electrode to form an electric field. A delivery component has a cargo carried thereby and is coupled with the first electrode such that the delivery component is capable of being positioned proximate to the target site. The delivery component is configured to degrade when exposed to the electric field formed between the first electrode and the second electrode so as to release the cargo to the target site.

Other aspects of the present invention relate to methods for delivering a cargo intraluminally to a target site of internal body tissue. The method includes disposing a first electrode proximate to a target site of internal body tissue, wherein the first electrode has a delivery component coupled thereto, and the delivery component is configured to carry a cargo therewith. The method further comprises opposably positioning a second electrode with respect to the first electrode such that the target site is disposed between the first and second electrodes. A voltage potential is applied across the first and second electrodes to form an electric field. In one aspect, the delivery component is configured to degrade when exposed to the electric field formed between the first electrode and the second electrode, thereby releasing the cargo. In another aspect, the electric field iontophoretically drives the cargo into the target site. In one aspect, the delivery component is configured to degrade when exposed to the electric field formed between the first electrode and the second electrode, and the delivery component is further configured to carry the charged cargo such that the charged cargo is iontophoretically delivered to the target site upon degradation thereof. In another aspect, the target site is removed from a first body location so as to externally receive the cargo in an ex vivo manner and transplanted to a second body location after receiving the cargo.

As such, embodiments of the present invention are provided to enable a highly targeted and efficient delivery of various cargos to predetermined target sites.

### BRIEF DESCRIPTION OF THE DRAWINGS

In order to assist the understanding of embodiments of the invention, reference will now be made to the appended drawings, which are not necessarily drawn to scale. The drawing is exemplary only, and should not be construed as limiting the invention.

FIG. 1 is a partial view of a delivery apparatus according to one embodiment of the present disclosure;

FIG. 2 is a partial view of a delivery apparatus according to one embodiment of the present disclosure, illustrating a delivery component capable of degradation to release a cargo to a target site;

FIG. 3 illustrates the placement of a delivery apparatus, according to one embodiment of the present disclosure, in a heart chamber;

FIG. 4 is a partial view of a delivery apparatus, according to one embodiment of the present disclosure, positioned in a blood vessel for iontophoretically delivering a cargo through an expandable member;

FIG. 5 illustrates a delivery apparatus for ex vivo iontophoretic treatment according to one embodiment of the present disclosure;

FIG. 6 is a partial view of the delivery apparatus of FIG. 5;

FIGS. 7A and 7B are images illustrating implementation of a delivery apparatus according to one aspect of the present disclosure;

FIGS. 8A and 8B are images illustrating implementation of a delivery apparatus according to another aspect of the present disclosure;

FIGS. 9A and 9B are images illustrating implementation of a delivery apparatus according to yet another aspect of the present disclosure; and

FIGS. 10A and 10B are images illustrating implementation of a delivery apparatus according to still another aspect of the present disclosure.

### DETAILED DESCRIPTION OF THE PREFERRED EMBODIMENTS

Embodiments of the present inventions now will be described more fully hereinafter with reference to the accompanying drawings. The invention may be embodied in many different forms and should not be construed as limited to the embodiments set forth herein; rather, these embodiments are provided so that this disclosure will satisfy applicable legal requirements. Like numbers refer to like elements throughout.

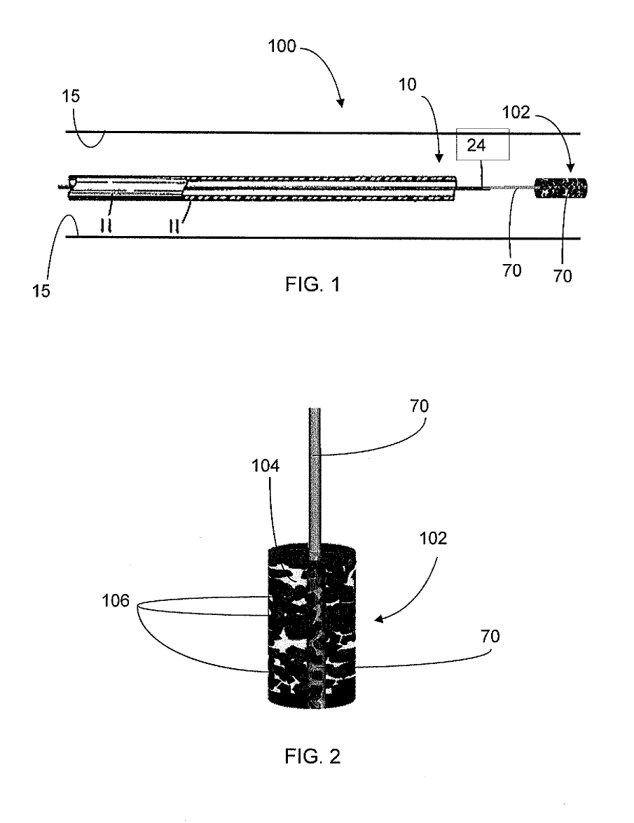

FIGS. 1-6 illustrate various embodiments of delivery apparatus in accordance with the present invention. In general, the delivery apparatus is provided for delivering a cargo to, or through, a localized area of a passageway in order to treat the localized area of the passageway or to treat a localized area of tissue located adjacent to the passageway, with minimal, if any, undesirable effect on other body tissue. Such an apparatus may be inserted intraluminally, through natural orifices, ex vivo or via direct injection. In some instances, the delivery apparatus may include a degradable delivery component for releasing the cargo in the localized area. In some instances, the delivery apparatus may include a delivery component that may be electrochemically degraded upon the flow of a current, thereby releasing the cargo to either diffuse into the surrounding tissue or, upon further application of an electric field, a charged cargo (i.e., a cargo being ionically charged) may be driven into the surrounding tissue by ionotophoretic techniques. In other instances, a modified catheter balloon design, which can be used in conjunction with existing catheters, may be used to encapsulate the degradable delivery component. The term catheter as used in the present application is intended to broadly include any medical device designed for insertion into a body passageway to permit injection or withdrawal of fluids, to keep a passage open or for any other purpose. In some instances, the term cargo refers to a particle that contains a therapeutic. In some instances, the term cargo refers to a therapeutic. A therapeutic can include a small molecule, biologic, or other substances utilized for the treatment or detection of disease. For example, the cargo can be a device that collects in a tumor bed to interact with tissue.

The delivery apparatus of the present invention has applicability for treating tissue and organ systems and, further, has applicability with any body passageway including, among others, blood vessels, tubular structures of the urinary, genitourinary, and intestinal tracts, the trachea and the like, and may be used to treat, for example, renal disease, uterine fibroids, urinary incontinence, erectile dysfunction, colorectal disease and inner and outer ear infections. Furthermore, other applications may include delivery of cargo for treating, for example, Parkinsons disease, stroke, and pain management. In other instances, the delivery apparatus may be implemented for delivery of therapeutic agents to the brain.

One particular application of the delivery apparatus may include the delivery of therapeutic agents to the cardiovascular system. Cardiovascular disease is the primary cause of death in the United States. The major underlying pathology of cardiovascular disease is atherosclerosis, which has manifestations ranging from narrowing of the coronary arteries due to plaque formation, to acute plaque rupture causing myocardial infarction. Coronary bypass surgery is a common treatment option wherein a vein, typically from the leg or chest cavity, is used to route blood around a blockage in the heart. Unfortunately this procedure has a high long-term failure rate due intimal hyperplasia and restenosis caused by vascular smooth muscle cell proliferation into the bypass. Restenosis is considered to be the “Achilles' heel” of percutaneous transluminal coronary angioplasty. Restenosis is a complex process of injury-induced events triggered by vessel wall damage.

Accordingly, embodiments of the present invention allow high concentrations of therapeutic agents to be delivered directly to the site of angioplasty without exposing the entire circulation to the medication and with the ability to protect delicate therapeutics such as, for example, siRNA from degradation while in circulation. Furthermore, embodiments of the present invention facilitate the delivery of therapeutics to the site of the vulnerable plaque prior to rupture.

FIG. 1 illustrates a delivery apparatus 100 which may deliver cargo iontophoretically to target sites for localized treatment. Iontophoresis technology is known in the art and is commonly used in transdermal drug delivery. In general, iontophoresis technology uses an electrical potential or current across a semipermeable barrier to drive ionic fixatives or drugs or drag nonionic fixatives or drugs in an ionic solution. Iontophoresis facilitates both transport of the fixative or drug across the selectively permeable membrane and enhances tissue penetration. In the application of iontophoresis, two electrodes, one on each side of the barrier, are utilized to develop the required potential or current flow. In particular, one electrode may be located inside of the catheter in opposed relation to the drug delivery wall of the catheter while the other electrode may be located at a remote site on a patient's skin.

FIG. 1 illustrates one particular embodiment of the delivery apparatus 100. In some instances, the delivery apparatus 100 may include a flexible catheter body 11. For example, such catheters are commonly used in percutaneous transluminal coronary angioplasty (PTCA) procedures to dilate stenosed blood vessels or arteries. Catheter 11 may be configured so as to be introduced the body through a guide catheter, or over a guide wire, or in another desirable manner. Catheter 11 may include an elongate portion with one or more electrodes 70 disposed thereon proximate to a distal end 10 thereof. The distal end 10 of catheter 11 is capable of insertion into an arterial vessel or other body passageway in which the vessel walls are indicated by the reference numeral 15, wherein the catheter 11 is extended through a vessel or other body passageway to be positioned proximate to the target site (i.e., the body tissue targeted for treatment or otherwise targeted for receipt of the cargo).

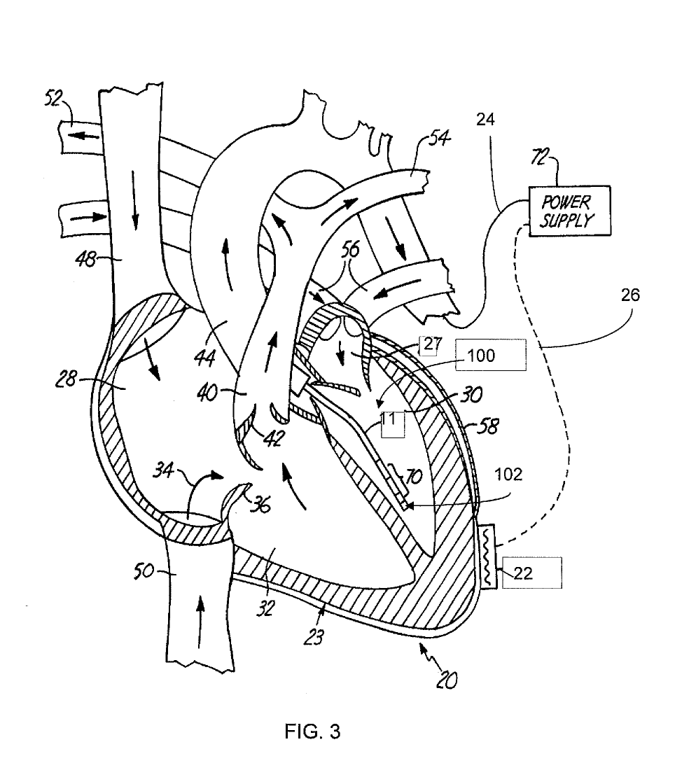

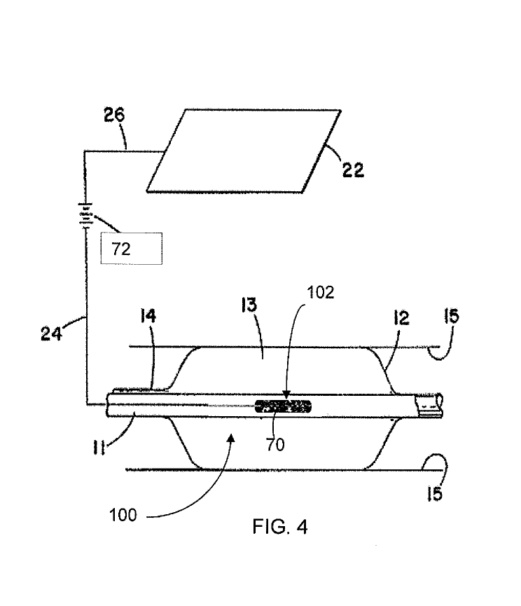

The catheter may include a delivery component 102 disposed near its distal end 10. In some embodiments, the delivery component 102, carrying an ionically charged cargo, may traverse the interior of the catheter to reach the target site so as to maintain the integrity of the delivery component 102. An electrical lead 24 may be provided so as to electrically connect the electrodes 70 to a power supply 72 (See FIGS. 3 and 4). A return electrode 22 (See FIGS. 3 and 4) may be positioned, for example, on the surface of the patient's body and connected to the power supply 72 by an electrical lead 26. In such instances, a voltage potential can be achieved between the electrodes 22, 70 such that the ionically charged cargo is repelled from the electrode 70 and attracted to the electrode 22 to promote deep penetration of the cargo into the body tissue. In some instances, return electrode 22 may have pressure-sensitive adhesive backing and low impedances at the skin to electrode interface. The preferred electrode materials should minimize undesired oxidative/reductive reactions or production of competitive ions during the iontophoresis. For example, electrode materials may include platinum or any other suitable materials or, in other instances, silver for anodal electrodes and silver/silver chloride for cathodal electrodes.

The delivery component 102 may, in some instances, be constructed of a degradable structure capable of being electrochemically degraded. In some instances, the delivery component 102 may be a polymer network/matrix, such as, for example, a hydrogel, which oxidatively breaks down due to the voltage at the electrode. As the polymer becomes soluble, the polymer and the cargo are released from the anode. The degradative network/matrix may facilitate quick and improved release of all cargo from the electrode. In other embodiments, the polymer may be a hydrogel which swells and releases the cargo so as to be delivered to the target site. Still, in other embodiments, the delivery component may include a polymer or sponge-type material capable of being saturated with a charged cargo. In some cases the degradable polymer may also be entrained within a semipermeable membrane to facilitate keeping the degradable polymer within close proximity of the electrode and lending mechanical stability to the materials.

In one exemplary embodiment of the present invention, as illustrated in FIG. 2, the delivery component 102 may generally be in contact with or otherwise coupled to the electrode 70 at the end or “tip” thereof, wherein the delivery component 102 may consist of cargo 104 physically entrapped in a crosslinked network/matrix 106 which is capable of degrading such that the network/matrix 106 falls apart once a voltage is applied, thereby releasing the cargo 104. In some instances the cargo 104 may be repelled from the like charge of the electrode 70. The delivery component may be structured to the electrode 70 in any suitable manner, such as, for example, a loop structure, which holds the network/matrix 106. In other embodiments, the network/matrix 106 may be encapsulated in a porous material such that the cargo 104, after degradation of the network/matrix 106, passes through the porous material to reach the target site. In some instances, the network/matrix 106 may be degraded by the cleavage of a vicinal diol. In such instances, the network/matrix 106 may be degraded by electrochemically breaking the carbon-carbon bond in a vicinal diol at a predetermined current and voltage, as supplied by power supply 72.

As an example, a hydrogel of acrylic acid and the sodium salt form of acrylic acid (0-50 wt %) may be crosslinked with a divinyl vincinal diol, wherein breakdown of the crosslinked network occurs when using a Pt anode when treated under conditions of 10V (3 mA) for 20 minutes, as represented by the following:

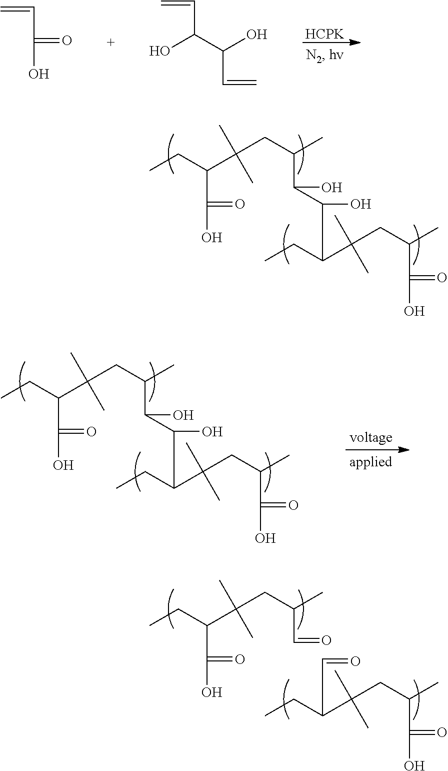

The cargo 104 may include small molecule ionic molecules, nucleic acids, proteins, therapeutic agents, diagnostic agents, and imaging agents as well as organic nanoparticles which may encapsulate a wide range of therapeutic, diagnostic, and imaging agents. The cargo may be configured to traffic preferentially based on size, shape, charge and surface functionality; and/or controllably release a therapeutic. Such cargos may include but are not limited to small molecule pharmaceuticals, therapeutic and diagnostic proteins, antibodies, DNA and RNA sequences, imaging agents, and other active pharmaceutical ingredients. Further, such cargo may include active agents wich may include, without limitation, analgesics, anti-inflammatory agents (including NSAIDs), anticancer agents, antimetabolites, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, therapeutic proteins, enzymes, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, xanthines, and antiviral agents. In addition, the cargo 104 may include a polynucleotide. The polynucleotide may be provided as an antisense agent or interfering RNA molecule such as an RNAi or siRNA molecule to disrupt or inhibit expression of an encoded protein.

Other cargo 104 may include, without limitation, MR imaging agents, contrast agents, gadolinium chelates, gadolinium-based contrast agents, radiosensitizers, such as, for example, 1,2,4-benzotriazin-3-amine 1,4-dioxide (SR 4889) and 1,2,4-benzotriazine-7-amine 1,4-dioxide (WIN 59075); platinum coordination complexes such as cisplatin and carboplatin; anthracenediones, such as mitoxantrone; substituted ureas, such as hydroxyurea; and adrenocortical suppressants, such as mitotane and aminoglutethimide.

In other embodiments, the cargo 104 may comprise Particle Replication In Non-wetting Templates (PRINT) nanoparticles (sometimes referred to as devices) such as disclosed, for example, in PCT WO 2005/101466 to DeSimone et al.; PCT WO 2007/024323 to DeSimone et al.; WO 2007/030698 to DeSimone et al.; and WO 2007/094829 to DeSimone et al., each of which is incorporated herein by reference. PRINT is a technology which produces monodisperse, shape specific particles which can encapsulate a wide variety of cargos including small molecules, biologics, nucleic acids, proteins, imaging agents. Cationically charged PRINT nanoparticles smaller than 1 micron are readily taken up by cells over a relatively short time frame, but penetration of the particles throughout the tissue is a longer process. For the delivery of PRINT nanoparticles throughout the tissue to be effective, the penetration needs to occur within a reasonable operational time frame. As such, the delivery apparatus 100 may be used to achieve such penetration by employing iontophoresis, in which charged PRINT nanoparticles are driven into body tissue using repulsive electromotive forces. The PRINT particles may or may not contain a therapeutic. In some instances, the cargo may be a therapeutic agent such as PLGA. In addition, the PRINT nanoparticles may be engineered to achieve a certain mission, and design-in handles that permit remote control for externally turning the cargo “on” or switching it “off”. As such, the cargo may be manipulated using ultrasound, low-dose radiation, magnetics, and other suitable means.

In other instances, the delivery apparatus 100 may be used to provide therapeutic treatment, for example, to heart tissue, as shown in FIG. 3, illustrating a partially sectioned view of a human heart 20 and its associated vasculature. The heart 20 is subdivided by muscular septum 22 into two lateral halves, which are named respectively right 23 and left 24. A transverse constriction subdivides each half of the heart into two cavities, or chambers. The upper chambers consist of the left and right atria 27, 28 which collect blood. The lower chambers consist of the left and right ventricles 30, 32 which pump blood. The arrows 34 indicate the direction of blood flow through the heart. The chambers are defined by the epicardial wall of the heart. The right atrium 28 communicates with the right ventricle 32 by the tricuspid valve 36. The left atrium 27 communicates with the left ventricle 30 by the mitral valve 38. The right ventricle 32 empties into the pulmonary artery 40 by way of the pulmonary valve 42. The left ventricle 30 empties into the aorta 44 by way of the aortic valve 46. The circulation of the heart 20 consists of two components. First is the functional circulation of the heart 20, i.e., the blood flow through the heart 20 from which blood is pumped to the lungs and the body in general. Second is the coronary circulation, i.e., the blood supply to the structures and muscles of the heart 20 itself.

The functional circulation of the heart 20 pumps blood to the body in general, i.e., the systematic circulation, and to the lungs for oxygenation, i.e., the pulmonic and pulmonary circulation. The left side of the heart supplies the systemic circulation throughout the rest of the body. The right side 23 of the heart supplies the lungs with blood for oxygenation. Deoxygenated blood from the systematic circulation is returned to the heart 20 and is supplied to the right atrium 28 by the superior and inferior venae cavae 48, 50. The heart 20 pumps the deoxygenated blood into the lungs for oxygenation by way of the main pulmonary artery 40. The main pulmonary artery 40 separates into the right and left pulmonary arteries, 52, 54 which circulate to the right and left lungs, respectively, oxygenated blood returns to the heart 20 at the left atrium 27 via four pulmonary veins 56 (of which two are shown). The blood then flows to the left ventricle 30 where it is pumped into the aorta 44, which supplies the body with oxygenated blood. The functional circulation, however, does not supply blood to the heart muscle or structures. Therefore, functional circulation does not supply oxygen or nutrients to the heart 20 itself. The actual blood supply to the heart structure, i.e., the oxygen and nutrient supply, is provided by the coronary circulation of the heart, consisting of coronary arteries, indicated generally at 58, and cardiac veins. Coronary artery 58 resides closely proximate the endocardial wall of heart 24.

With continuing reference to FIG. 3, the catheter 11 may be introduced into heart chamber 30. In such instances, the catheter 11 is introduced into left ventricle 30 through a guide catheter, or over a guide wire, or in another desirable manner. Catheter 11 may include an elongate portion with one or more electrodes 70 disposed thereon. Electrodes 70, which may comprise conductive sleeves or tabs, are coupled through a suitable conductor (wire) such as, for example, electrical lead 24 back through, or along side, catheter 11 to power supply 72. Delivery component 102 may be disposed at or otherwise proximate the tip of the electrodes 70, wherein the delivery component carries the cargo to be delivered to the tissue at the predetermined target site. Power supply 72 is energized to apply a constant low voltage to electrodes 70 to create either an anode or a cathode (depending upon the polarity of the cargo). Power supply 72 may have a second electrode 22 positioned external to the body (i.e., on the patient's skin) or, in other instances, within the body, wherein the second electrode 22 is connected to power supply 72 by electrical lead 26, which is indicated by dashed line 74.

In the illustrative embodiment, the cargo carried by delivery component 102 is energized to contain, for example, negative ions. When power supply 72 is energized, the delivery component 102 degrades and the electrodes 70 achieve a voltage potential with respect to the negatively charged ions of the cargo. The voltage potential created across the electrodes 70 and the electrode 22 sets up a field which interacts with the ionic cargo which acts to drive the charged cargo, such as a particle or therapeutic agent, into the heart tissue in the heart wall between the electrodes 70 and the electrode 22. That is, when the voltage potential is set up across electrodes 70 and 22, the ions of the cargo tend to migrate toward electrode 22. This drives the ions into the heart tissue between the electrodes 70 and 22. This driving force is the result of the iontophoretic technique. In some instances, electrode 22 may be inserted as a patch through a small hole in the chest and unfolded and then applied to the heart muscle. In any manner, the drug to be transferred to the heart muscle is provided on one side of the heart tissue. The electrode on the other side of the heart tissue is then energized to create the necessary field for transfer of the cargo into the heart tissue.

FIG. 4 illustrates embodiments of the present invention which are provided to deliver a cargo to a localized area of internal body tissue. As such, in some embodiments, the delivery apparatus 100 may include a flexible catheter connected to an expandable component having a fluid delivery passageway with an outer wall and selectively permeable outer membrane portion through which a cargo passes to an internal body tissue target site. For example, delivery apparatus may include a balloon component 12, as schematically shown in FIG. 4, which illustrates the distal end of the catheter 11 with the modified balloon component 12 in its inflated/expanded state. As described in previous embodiments, the catheter 11 may include a guide wire for positioning the catheter 11 near the target site, wherein its distal end and a balloon lumen or passageway 14 extend along the catheter 11 to the proximal end of the catheter 11 for inflation and deflation of the balloon component 12. In some embodiments, the material from which the balloon 12 is constructed is a permeable or semi-permeable material which is effective to permit transport or passage of the cargo across the balloon surface as a result of iontophoresis according to the present disclosure. The balloon component may, in some instances, encapsulate the delivery component 102, wherein the delivery component is engaged with the at least one of the electrodes 70. The balloon component 12 may, in some embodiments, have the following characteristics: perfusion balloon design to allow for distal flow during inflation; a low profile design; highly compliant, low modulus balloon material; and operates at relatively low pressures.

In one particular embodiment, as illustrated in FIG. 4, the delivery apparatus 100 may comprise balloon component 12 that is provided in its inflated state within an arterial vessel in which the vessel walls are indicated by the reference numeral 15. During intravessel procedures, such as PTCA, the guide wire (not shown) is first inserted into the selected artery to a point past the stenotic lesion. The catheter 11 may then be advanced along the guide wire to the desired position or site in the arterial system in which the balloon component 12 traverses or crosses the stenotic lesion. The balloon component 12 is then inflated by introducing an inflation fluid through the balloon lumen 14 into the interior chamber 13 of the balloon component 12. During inflation, the outer surfaces of the balloon component 12 press outwardly against the inner surfaces of the vessel wall 15 to expand or dilate the vessel in the area of the stenotic lesion. In some instances, the balloon 12 may be inflated by introducing a fixation or other drug solution through the balloon lumen 14 and into the interior of the balloon portion 12. In some embodiments, the balloon component 12 may inflate once in a single location and deploy the cargo into the surrounding tissue. In other embodiments, the balloon component 12 may mechanically inflate multiple times in several locations, delivering therapeutics or particles at each location.

The embodiment of FIG. 4 illustrates a structure utilizing iontophoresis to assist in driving the cargo across the balloon wall 12 and into contact with the vessel walls 15. The electrode 70 may be located on or within the catheter body 11 while the other electrode 22, the body surface electrode, is located on the body surface (i.e., a patch applied to the patient's skin) or within the body of the patient. In order for iontophoresis techniques to be utilized, the cargo within the balloon chamber 13 requires specific characteristics. Such cargo may have an ionic nature or have other ionic molecules bound to the cargo to promote the iontophoretic movement or transport across the balloon wall 12. An electrical current is produced between the electrodes 70 and 22 by the external power source 72 through the electrical leads 24 and 26, respectively. During operation of the delivery apparatus 100, the balloon component 12 may be first positioned across the stenotic lesion in the manner described above. The balloon interior 13 is then inflated with the fixative through the lumen 14. This is followed by activating the power supply 72, thereby creating a net flow of current between electrode 70 and electrode 22 which passes through the balloon wall 12. As previously described, the current flow causes degradation of the delivery component 102 disposed within the balloon component 12 so as to facilitate the controllable release of the cargo carried therewith. The released cargo can diffuse in contact with the surrounding vessel wall 15 and vascular tissue. In some instances, the net current flow drives or drags the cargo, now released/deployed, within the chamber 13 across the wall and into contact with the surrounding vessel wall 15 and vascular tissue. The delivery apparatus 100 may utilize both pressure and iontophoresis as the driving force, although, it is envisioned that iontophoresis could be utilized alone.

In some embodiments, the balloon component 12 may be constructed of a various inflatable/expandable substrates that may be permeable, microporous or semi-permeable materials, which may include, for example, ePTFE, VTEC, nitinol, cellulose, cellulose acetate, polyvinyl chloride, polysulfone, polyacrylonitrile, silicon, polyurethanes, natural and synthetic elastomers, polyester, polyolefin, a fluorpolymer, or any other suitable material.

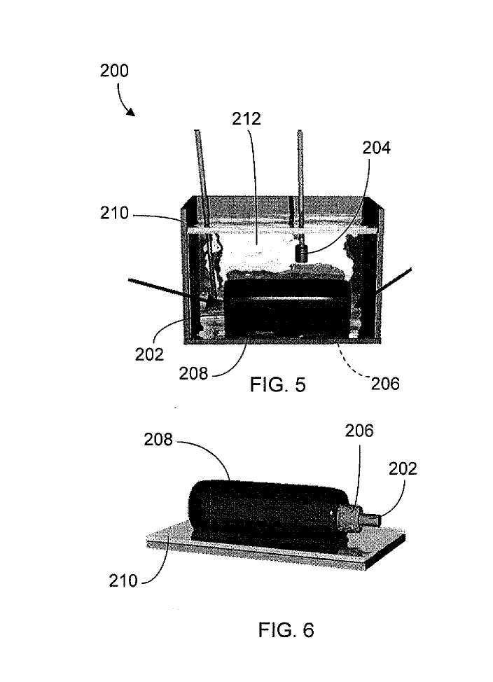

In other instances, the delivery apparatus 100 may be applied in an ex vivo manner, in which, for example, the delivery apparatus 100 is used for therapeutic delivery to vein segments which are removed from one location in the patient and transplanted to another location. That is, a bodily portion, such as a vein segment, may be removed from the body for treatment and then transplanted to a different location within the body thereafter. For example, the delivery apparatus 100 may be utilized for pre-treatment of arteries/veins harvested from the legs/arms (of the patient or of a cadaver or other model), for transplant into other regions of the body. As shown in FIGS. 5 and 6, embodiments of such a delivery apparatus 200 may include an anode 202 and a cathode 204 being electrically connected to a power supply (not shown). The opposing end of the anode not being connected to the power supply may have a delivery component 206, such as a polymer or sponge (saturated with a charged cargo), disposed thereabout, wherein the anode 202 and the delivery component 206 are disposed within a bodily portion 208. The bodily portion 208, anode 202, and delivery component 206 may be positioned in a container 210, wherein the container is filled with an electrically conductive media or solution 212, such as phosphate-buffered saline (PBS), such that the components within the container 210 are submerged therein. The cathode 204 may also be submerged within the PBS solution, wherein the cathode 204 may be positioned proximate to the bodily portion 208, externally with respect to the anode 202, such that the bodily portion 208 is positioned therebetween. In operation, an iontophoretic technique may be used, in which a voltage potential may be applied across the anode 202 and cathode 204 such that the charged cargo of the delivery component 206 is repelled by the anode 202 and attracted by the cathode 204 so as to deliver and drive the cargo into the bodily portion 208. One of ordinary skill in the art will recognize that the anode and cathode may be switched and that the cargo will be appropriately charged such that the cargo migrates toward and within the bodily portion.

In other embodiments of the present invention, placement of the cargo, such as the PRINT nanoparticles, may be achieved by using a needle having an iontophoretic tip to facilitate distribution of the particles into the surrounding target site (tissue). In such embodiments, the needle tip may represent a first electrode, while a second electrode is positioned external to the body so as to create a voltage potential when a power supply is energized, as described previously with respect to iontophoretic techniques. Such a technique may be used for disease states including cancer (brain, prostate, colon, others), inflammation, damaged tissue ‘rescue’ situations (e.g. cardio/neuro/peripheral vascular), ocular diseases, rhinitis, and other applications. Still, in other embodiments, placement of the cargo, such as PRINT nanoparticles, may be achieved using endovascular or NOTES-based devices, for the minimally invasive treatment of accessible cancers. Such treatment may include colon, pancreatic, brain, esophageal, liver, cervical, and ovarian. These devices may be passive in nature (elution or simple placement), or may be more active in placement method (iontophoretic, ultrasound, radio/micro waves).

Many modifications and other embodiments of the invention will come to mind to one skilled in the art to which this invention pertains having the benefit of the teachings presented in the foregoing description; and it will be apparent to those skilled in the art that variations and modifications of the present invention can be made without departing from the scope or spirit of the invention. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

The following examples are presented by way of illustration, not by way of limitation.

### EXPERIMENTAL

### Example 1

### Dye Delivery in a Mock Vessel

A tube of agarose gel measuring 2.5 cm in length with an outer diameter of 1.5 cm and an inner diameter of 0.5 cm was used as a mock vessel. Covered copper electrical wiring was used and the electrode consisted of a stripped end of the wire. A piece of sponge approximately 2 cm in length and 0.5 cm in diameter was placed over the stripped end of one piece of copper electrical wire which was to be the anode.

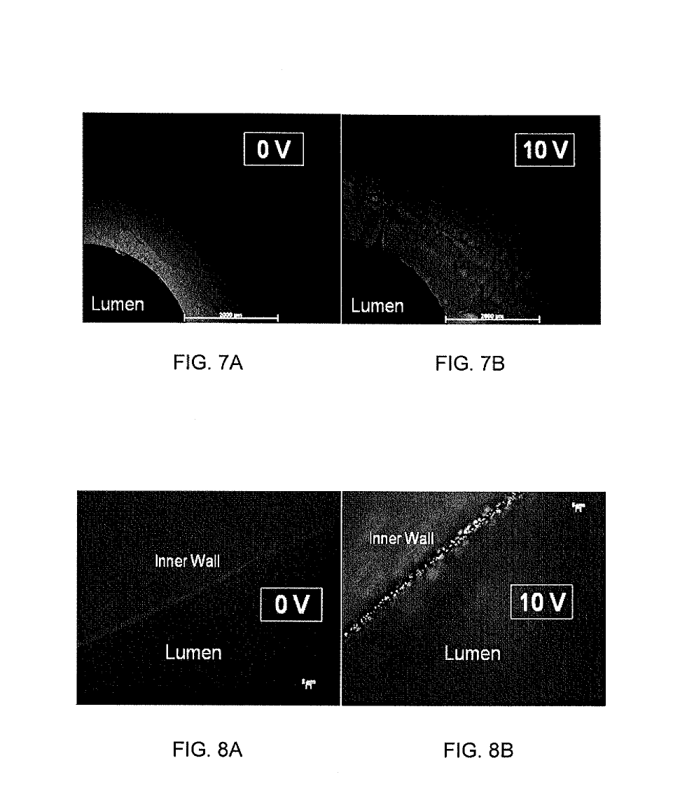

The sponge was thoroughly soaked in a solution of Rhodamine B, a cationic dye, in water. The sponge was then placed inside the agarose vessel and the other end of the wire was hooked to the anode of a DC power source with an alligator clip. The agarose vessel was submersed in a polypropylene dish containing PBS. The cathode, a second piece of copper wire, was placed in the PBS beside the agarose vessel. In the negative control, this soaked without voltage for 10 minutes. In the experimental condition, the voltage applied was 10V and the current was 22 mA. This also ran for 10 minutes. To characterize, a cross-section of the agarose vessel was taken and fluorescence microscopy was used, as shown in FIGS. 7A and 7B. The magnification, scale bar, placement of vessel, and camera shutter setting were held constant. In the negative control (0V), dye is localized to the inner wall, while in the experimental condition (10V) the dye has spread into the vessel.

### Example 2

### Particle Delivery in a Mock Vessel

A tube of agarose gel measuring 2.5 cm in length with an outer diameter of 1.5 cm and an inner diameter of 0.5 cm was used as a mock vessel. Covered copper electrical wiring was used and the electrode consisted of a stripped end of the wire. A piece of sponge approximately 2 cm in length and 0.5 cm in diameter was placed over the stripped end of one piece of copper electrical wire which was to be the anode.

The sponge was soaked in a solution of 1 micron cationically charged particles tagged with FITC. The same procedure used in Example 1 was followed. The difference, as shown in FIGS. 8A and 8B, is that in the case of the negative control (0V) there are no particles on the inner wall of the agarose vessel while in the experimental condition (10V) the inner wall of the agarose vessel is covered with particles. Thus, the particles are repelled from the anode towards the inner wall.

### Example 3

### Particle Delivery in a Pig Splenic Artery with Steady Voltage

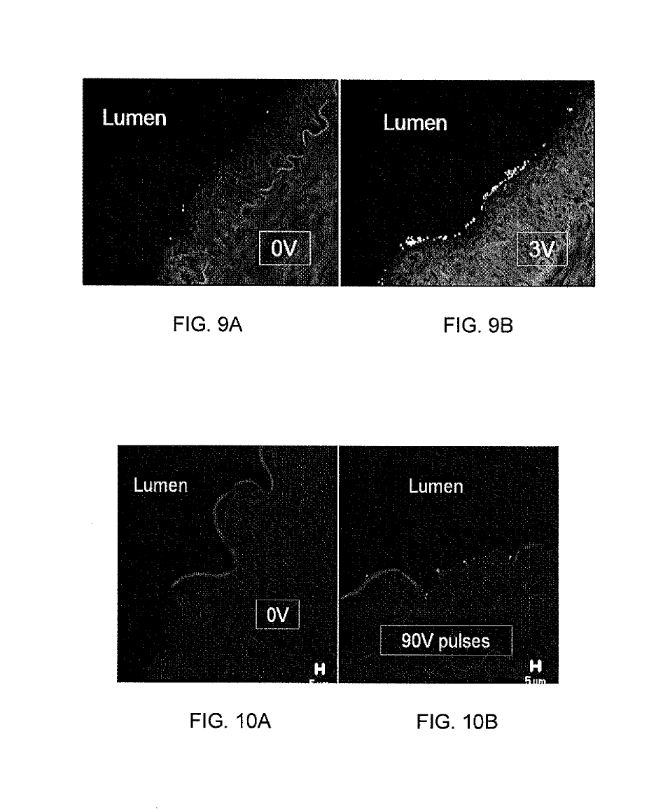

The splenic artery of a pig was excised and cut into sections approximately 1 cm long. Particles were made using the PRINT® technology. A monomer solution consisting of 88% poly(ethylene glycol) triacrylate, 10% [2-(acryloyloxy)ethyl]trimethylammonium chloride, 1% fluorescein-o-acrylate, and 1% diethoxyacetophenone was used to fill a 2 micron cubic mold and photocured. These particles were then collected. The solution of cationic particles was injected into the luminal space of the artery. A silver wire measuring 0.125 mm in diameter acted as the anode and was inserted into the luminal space and attached to a DC power source. The artery was placed in a water bath. The cathode, a second piece of silver wire, was placed beside the artery. In the control no voltage was applied. In the experimental condition 3V was applied for 5 minutes. The vessels were fixed and histology slices were prepared. Fluorescent microscopy was used to image the histology sections. As shown in FIGS. 9A and 9B, application of voltage resulted in a much higher accumulation of particles on the vessel wall just as was seen with particles in a mock vessel (Example 2). Given time, these particles could be taken up by the endothelial cells lining the artery wall.

### Example 4

### Particle Delivery in a Dog Carotid Artery with Pulsed Voltage

The carotid artery of a dog was excised and cut into sections approximately 1 cm long. Particles were made using the PRINT® technology. A monomer solution consisting of 65% poly(ethylene glycol) triacrylate, 20% poly(ethylene glycol) monomethacrylate, 10% amino-ethylmethacrylate, 3% fluorescein-o-acrylate, and 2% diethoxyacetophenone was used to fill a 200 nanometer cylindrical mold and photocured. These particles were then collected. The solution of cationic particles was injected into the luminal space of the artery. A silver wire measuring 0.125 mm in diameter acted as the anode and was inserted into the luminal space and attached to a DC power source. The vessel was placed in a water bath. The cathode, a second piece of silver wire, was placed beside the artery. In the control, no voltage was applied. In the experimental condition, 90V pulses approximately 1 second in duration every 5 seconds were applied for 1 minute. The vessels were fixed and histology slices were prepared. As shown in FIGS. 10A and 10B, application of voltage resulted in a higher accumulation of particles on the vessel wall though not as high as achieved with steady voltage (Example 3).

What is claimed is:

1. An apparatus adapted for delivering a cargo to a target site of internal body tissue, comprising:
a flexible tubular member having a distal end adapted for insertion proximate to a target site of internal body tissue;
a first electrode configured to extend within the flexible tubular member so as to be disposed proximate to the target site;
a second electrode in electrical communication with the first electrode and opposably positionable with respect thereto, the second electrode being configured to cooperate with the first electrode to form an electric field; and
a delivery component having a cargo carried thereby, the delivery component being coupled with the first electrode such that the delivery component is capable of being positioned proximate to the target site, and the delivery component being configured to degrade when exposed to the electric field formed between the first electrode and the second electrode so as to release the cargo to the target site.

2. An apparatus according to claim 1, wherein the delivery component comprises a polymer matrix material capable of electrochemical degradation when a voltage potential is applied across the first and second electrodes, wherein the cargo is released upon degradation of the delivery component.

3. An apparatus according to claim 1, wherein electric field formed by the first and second electrodes is capable of iontophoretically directing the cargo toward the target site so as to facilitate penetration of the cargo therein.

4. An apparatus according to claim 1, further comprising an expandable structure operably engaged with the first electrode, the expandable structure being configured to receive the delivery component therein such that, upon degradation thereof, the charged cargo is delivered to the target site.

5. An apparatus according to claim 4, wherein the expandable structure is configured to expand so as to contact the target site, and the expandable structure comprises a semi-permeable polymer material configured to permit the charged cargo to pass therethrough for delivery to the target site.

6. An apparatus according to claim 4, wherein the expandable structure is configured to both expand and deflate such that the expandable structure is capable of moving to more than one target site.

7. An apparatus according to claim 4, wherein the expandable structure comprises at least one of ePTFE, VTEC and nitinol.

8. An apparatus according to claim 1, wherein the charged cargo comprises at least one of small molecule, ionic molecules, nucleic acids, proteins, organic nanoparticles, therapeutic agents, and imaging agents.

9. An apparatus according to claim 1, wherein the second electrode comprises a patch member configured to be applied externally to the body such that the first electrode and the second electrode are positioned on opposing sides of the internal body tissue to which the cargo is to be delivered.

10. An apparatus according to claim 1, further comprising a porous structure operably engaged with at least one of the electrode and the flexible tubular member, the porous structure being configured to encapsulate the delivery component.

11. A method of delivering a cargo to a target site of body tissue, the method comprising:
disposing a first electrode proximate to a target site of internal body tissue, the first electrode having a delivery component coupled thereto, the delivery component being configured to carry a cargo therewith;
opposably positioning a second electrode with respect to first electrode such that target site is disposed between the first and second electrodes;
applying a voltage potential across the first and second electrodes to form an electric field; and
degrading the delivery component so as to release the cargo to the target site.

12. A method according to claim 11, further comprising iontophoretically driving the cargo into the target site by configuring the first electrode to repel the cargo and configuring the second electrode to attract the cargo.

13. A method according to claim 11, wherein disposing a first electrode proximate to a target site further comprises inserting a distal end of a flexible tubular member proximate to the target site, and extending the first electrode within the flexible tubular member to about the distal end thereof such that the first electrode and the delivery component are proximately disposed thereto.

14. A method according to claim 11, further comprising removing the target site from a first body location so as to externally receive the cargo, and further comprising transplanting the target site to a second body location after externally receiving the cargo.

15. A method according to claim 11, wherein operably engaging a delivery component to the first electrode further comprises operably engaging a delivery component being configured to degrade when exposed to the electric field formed between the first electrode and the second electrode, and further wherein the cargo carried by the delivery component is capable of being electrically charged such that the cargo is iontophoretically delivered to the target site upon degradation of the delivery component.

16. A method according to claim 11, wherein engaging a delivery component to the first electrode further comprises engaging a delivery component being encapsulated by a porous member such that the cargo is capable of passing through the porous member to contact a surface of the target site.

17. A method according to claim 11, wherein engaging a delivery component to the first electrode further comprises engaging a delivery component comprising a porous member capable of carrying the cargo by saturation thereof, and further wherein the porous member is configured to be expandable.

18. A method according to claim 11, wherein iontophoretically driving the cargo into the target site further comprises iontophoretically driving the cargo having an electric charge such that the cargo is repelled from the first electrode and attracted to the second electrode so as to facilitate penetration of the surface of the target site.

19. A method according to claim 11, wherein opposably positioning a second electrode proximate to the target site such that the target site is positioned between the first and second electrodes further comprises positioning a second electrode externally to a body member.
